(19) United States
(12) Patent Application Publication
(10) Pub. No.: US 2022/0211993 A1
(43) Pub. Date: Jul. 7, 2022

(54) MEDICAL DISINFECTANT CAPS

(71) Applicant: Codan US Corporation, Santa Ana, CA (US)

(72) Inventor: Thomas F. Knight, Santa Ana, CA (US)

(73) Assignee: Codan US Corporation, Santa Ana, CA (US)

(21) Appl. No.: 17/143,926

(22) Filed: Jan. 7, 2021

(51) Int. Cl. A61M 39/20; A61M 39/22; A61M 39/16

(52) U.S. Cl. CPC A61M 39/20; A61M 2039/1066; A61M 39/16; A61M 39/22

(57) ABSTRACT

Caps for disinfecting medical devices, and methods of disinfecting medical device using the caps, are disclosed. In general, one aspect disclosed features a cap for disinfecting a medical device, the cap comprising: a cylindrical receiving portion having an opening configured to receive a medical device, the cylindrical receiving portion comprising internal threads configured to mate with external threads of the medical device; disinfectant disposed within the cylindrical receiving portion; a cylindrical stem disposed longitudinally within the cylindrical receiving portion; and an annular sponge disposed about the cylindrical stem and contacting the disinfectant.

### DESCRIPTION OF RELATED ART

The disclosed technology relates generally to medical devices, and more particularly, some embodiments relate to systems and methods for such devices.

### SUMMARY

In general, one aspect disclosed features a cap for disinfecting a medical device, the cap comprising: a cylindrical receiving portion having an opening configured to receive a medical device, the cylindrical receiving portion comprising internal threads configured to mate with external threads of the medical device; disinfectant disposed within the cylindrical receiving portion; a cylindrical stem disposed longitudinally within the cylindrical receiving portion; and an annular sponge disposed about the cylindrical stem and contacting the disinfectant.

Embodiments of the cap may include one or more of the following features. In some embodiments, the annular sponge is made of polyurethane. In some embodiments, the annular sponge is approximately ¼ inch thick. Some embodiments comprise one or more longitudinal grooves formed in the cylindrical stem. Some embodiments comprise a removable seal configured to retain the disinfectant within the cylindrical receiving portion. In some embodiments, the medical device comprises one of: a female luer lock; or a needleless valve.

In general, one aspect disclosed features a cap for disinfecting a medical device, the cap comprising: a cylindrical receiving portion having an opening configured to receive a medical device; a cylindrical stem disposed longitudinally within the cylindrical receiving portion; and disinfectant disposed within the cylindrical receiving portion.

Embodiments of the cap may include one or more of the following features. In some embodiments, the cylindrical receiving portion comprises: internal threads configured to mate with external threads of the medical device. Some embodiments comprise an annular sponge disposed about the cylindrical stem and contacting the disinfectant. In some embodiments, the annular sponge is made of polyurethane. In some embodiments, the annular sponge is approximately ¼ inch thick. Some embodiments comprise one or more longitudinal grooves formed in the cylindrical stem. Some embodiments comprise a removable seal configured to retain the disinfectant within the cylindrical receiving portion. In some embodiments, the disinfectant is 70/30 isopropyl alcohol. In some embodiments, the medical device comprises one of: a female luer lock; or a needleless valve.

In general, one aspect disclosed features a method of disinfecting a medical device, the method comprising: providing a medical device; providing a cap, the cap comprising: a cylindrical receiving portion having an opening configured to receive a medical device, a cylindrical stem disposed longitudinally within the cylindrical receiving portion, and disinfectant disposed within the cylindrical receiving portion; and inserting the medical device into the cylindrical receiving portion of the cap such that the disinfectant contacts the medical device. Embodiments of the method may include one or more of the following features. In some embodiments, the cylindrical receiving portion comprises internal threads configured to mate with external threads of the medical device, the method further comprising: turning the cap with respect to the medical device such that the internal threads of the cap engage external threads of the medical device. In some embodiments, the cap comprises an annular sponge disposed about the cylindrical stem and contacting the disinfectant, the method further comprising: turning the cap with respect to the medical device such that the internal threads of the cap engage external threads of the medical device and the annular sponge is compressed to release the disinfectant. Some embodiments comprise removing the medical device from the cylindrical receiving portion of the cap; and allowing the disinfectant to dissipate from the medical device. In some embodiments, the medical device comprises one of: a female luer lock; or a needleless valve.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 is an illustration of a medical disinfectant cap according to some embodiments of the disclosed technology.

FIG. 2 is an illustration of an annular sponge according to some embodiments of the disclosed technology.

FIG. 3 is an illustration of medical disinfecting cap having a grooved stem according to some embodiments of the disclosed technology.

FIG. 4 illustrates a removable seal according to some embodiments of the disclosed technology.

FIG. 5 illustrates a sealed cap according to some embodiments of the disclosed technology.

FIG. 6 illustrates a medical disinfectant cap and a 3-way stopcock having a female luer lock prior to connecting the cap to the stopcock.

FIG. 7 illustrates the medical disinfectant cap as connected to the 3 way stopcock.

FIGS. 8-10 are cross-sections of the medical disinfectant cap and the female luer lock of FIGS. 6 and 7.

In FIG. 8, the cap and the female luer lock have been brought into contact, but their threads have not engaged.

In FIG. 9, the cap has been threaded partially onto the female luer lock, and the female luer lock has made contact with the sponge.

In FIG. 10, the cap has been threaded fully onto the female luer lock, which has compressed the sponge, thereby releasing disinfectant to bathe and disinfect the exterior surfaces of the female luer lock.

FIG. 11 illustrates a medical disinfectant cap and a 3-way stopcock having a needleless valve prior to connecting the cap to the stopcock.

FIG. 12 illustrates the medical disinfectant cap as connected to the 3 way stopcock 1104.

FIGS. 13-15 are cross-sections of the medical disinfectant cap and the needleless valve of FIGS. 11 and 12.

In FIG. 13, the cap and the needleless valve have been brought into contact, but their threads have not engaged.

In FIG. 14, the cap has been threaded partially onto the needleless valve 1106, and the needleless valve has made contact with the sponge 1302.

In FIG. 15, the cap has been threaded fully onto the needleless valve, which has compressed the sponge, thereby releasing disinfectant to bathe and disinfect the exterior surfaces of the needleless valve, including the surface of the silicone insert of the needleless valve.

FIG. 16 is a flowchart illustrating a process for disinfecting a medical device according to some embodiments of the disclosed technologies.

The figures are not exhaustive and do not limit the present disclosure to the precise form disclosed.

### DETAILED DESCRIPTION

It is often desirable to administer fluids to patients intravenously. Intravenous fluid delivery systems often include connectors that allows the patient to leave the fluid delivery system temporarily, for example when the patient would like to visit the bathroom. But while disconnected, the connectors are exposed to the environment, and may become contaminated. To reduce the risk of contamination, the connectors are usually disinfected before reconnection. For example, the connectors may be disinfected using a swab containing a disinfectant fluid.

Embodiments of the disclosed technology provide medical disinfectant caps for disinfecting medical devices, and methods for using the medical disinfectant caps to disinfect medical devices. The disclosed medical disinfectant caps may include internal threads for mating with the external threads of a medical device. The caps may include disinfectant. A medical device may be disinfected by simply threading the cap onto the medical device, thereby allowing the disinfectant to contact and disinfect the medical device. The disinfectant may be 70/30 isopropyl alcohol. In the disclosed embodiments, the kill levels achieved may match or exceed competition kill levels.

In some embodiments, the caps include a sponge soaked with the disinfectant. In these embodiments, threading the cap onto the medical devices compresses the sponge, thereby releasing the disinfectant to contact and disinfect the medical device.

The caps may include a cylindrical stem disposed longitudinally within the cap. The stem may be configured to prevent the disinfectant from entering an interior space of the medical device, while allowing the disinfectant to bathe exterior surfaces of the medical device. These embodiments are especially useful for disinfecting female luer locks, and similar medical devices.

In some embodiments having a stem, one or more longitudinal grooves may be formed in the stem to allow the disinfectant to flow past the stem. These embodiments are especially useful for disinfecting needleless valves, and similar medical devices.

In embodiments having a stem, the sponge soaked with disinfectant may be annular and disposed about the cylindrical stem. In some embodiments, the caps may be sealed with a removable seal to retain the disinfectant until use.

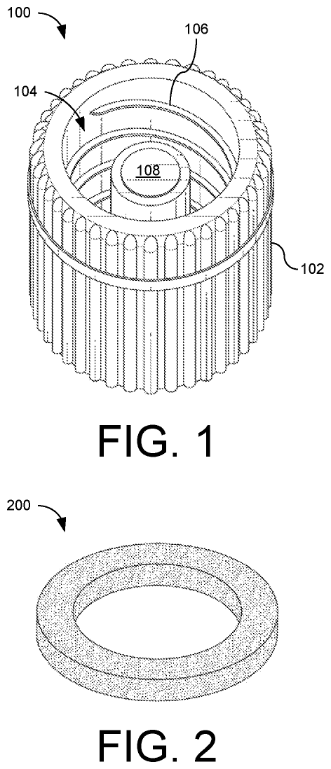

FIG. 1 is an illustration of a medical disinfectant cap 100 according to some embodiments of the disclosed technology. Referring to FIG. 1, the cap 100 may include a cylindrical receiving portion 102 having an opening 104 configured to receive a medical device. The cylindrical receiving portion 102 may include internal threads 106. The internal threads 106 may be configured to mate with external threads of a medical device.

The cap 100 may include a cylindrical stem 108 disposed longitudinally within the cylindrical receiving portion 102. The cylindrical stem 108 may prevent the disinfectant from entering the interior of a medical device such as a female luer lock.

In some embodiments, the cap 100 may include an annular sponge. FIG. 2 is an illustration of an annular sponge 200 according to some embodiments of the disclosed technology. The annular sponge 200 may be made of polyurethane, or similar materials. In some embodiments, the annular sponge is approximately ¼ inch thick. The annular sponge 200 may be disposed about the cylindrical stem 108, and may be soaked with disinfectant.

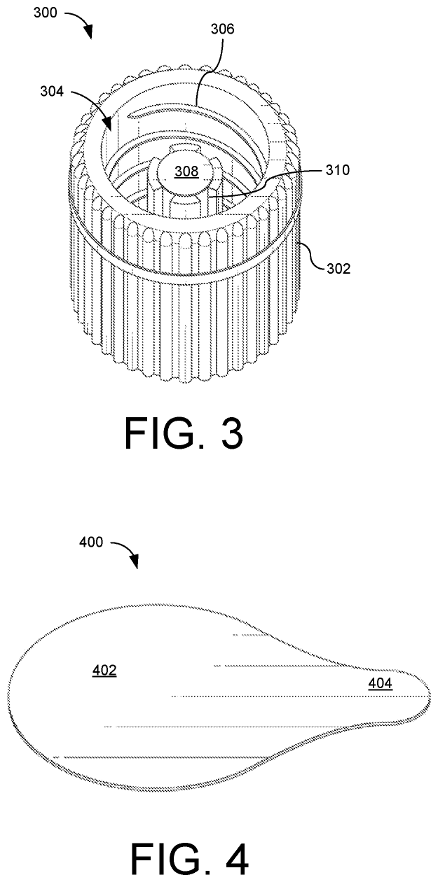

In some embodiments, the stem of the cap may include one or more longitudinal grooves. FIG. 3 is an illustration of medical disinfecting cap 300 having a grooved stem according to some embodiments of the disclosed technology. Referring to FIG. 3, the cap 300 may include a cylindrical receiving portion 302 having an opening 304 configured to receive a medical device. The cylindrical receiving portion 302 may include internal threads 306. The internal threads 306 may be configured to mate with external threads of a medical device. The cap 300 may include a cylindrical stem 308 disposed longitudinally within the cylindrical receiving portion 302. The cap 300 may include an annular sponge such as the annular sponge 200 of FIG. 2. The annular sponge may be something disinfectant. The cap 300, sponge, and disinfectant, may be sealed using a removable seal such as the removable seal 400 of FIG. 4.

The cylindrical stem 308 may include one or more longitudinal grooves 310. The grooves 310 may allow the disinfectant to flow past the stem 308 to bathe an interior surface of a medical device such as a needless valve, and similar medical devices.

In some embodiments, the cap 100 may be sealed with a removable seal prior to use. FIG. 4 illustrates a removable seal 400 according to some embodiments of the disclosed technology. Referring to FIG. 4, the removable seal 400 may include a cylindrical body portion 402 for sealing the opening 104 of the cap 100, as well as a tab portion 404 that may be grasped to facilitate removal of the removable seal 400. The removable seal 400 may be made of aluminum, or similar materials.

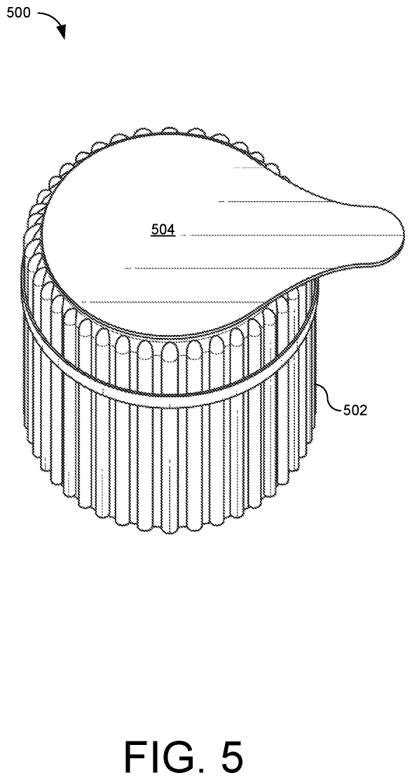

FIG. 5 illustrates a sealed cap according to some embodiments of the disclosed technology. Referring to FIG. 5, a cap 502 is sealed with a removable seal 504. The cap 502 may be implemented as the cap 100 of FIG. 1, the cap 300 of FIG. 3, or other embodiments of the disclosed caps. The removable seal 504 may be implemented as the removable seal 400 of FIG. 4, or other embodiments of the disclosed removable seals.

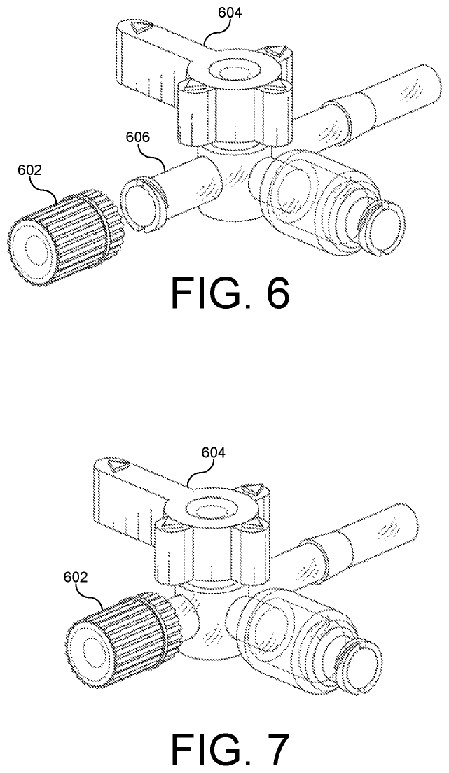

FIG. 6 illustrates a medical disinfectant cap 602 and a 3-way stopcock 604 having a female luer lock 606 prior to connecting the cap 602 to the stopcock 604. The cap 602 may be implemented for example as the cap 100 of FIG. 1. The stem of the cap 602 may have no grooves, and therefore when connected prevents the disinfectant in the cap 602 from entering the interior of the stopcock 604. FIG. 7 illustrates the medical disinfectant cap 602 as connected to the 3 way stopcock 604.

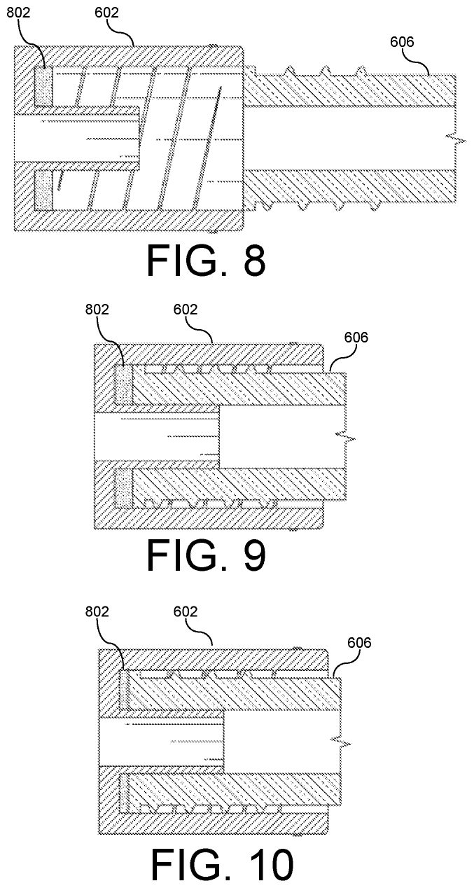

FIGS. 8-10 are cross-sections of the medical disinfectant cap 602 and the female luer lock 606 of FIGS. 6 and 7. In each of FIGS. 8-10, an annular sponge 802 is disposed within the cap 602. In FIG. 8, the cap 602 and the female luer lock 606 have been brought into contact, but their threads have not engaged.

In FIG. 9, the cap 602 has been threaded partially onto the female luer lock 606, and the female luer lock 606 has made contact with the sponge 802.

In FIG. 10, the cap 602 has been threaded fully onto the female luer lock 606, which has compressed the sponge 802, thereby releasing disinfectant to bathe and disinfect the exterior surfaces of the female luer lock 606.

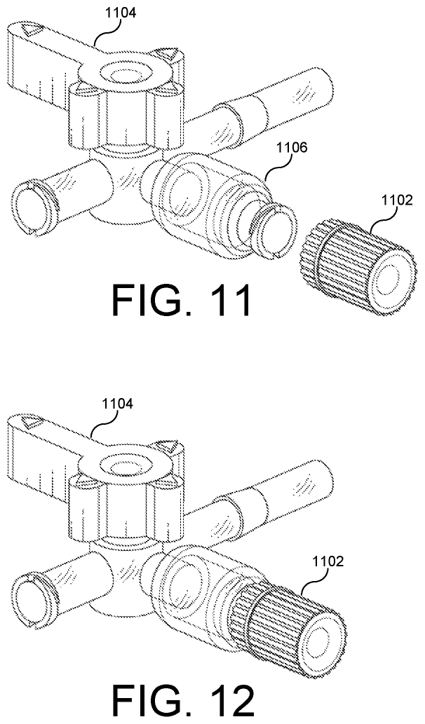

FIG. 11 illustrates a medical disinfectant cap 1102 and a 3-way stopcock 1104 having a needleless valve 1106 prior to connecting the cap 1102 to the stopcock 1104. The cap 1102 may be implemented for example as the cap 300 of FIG. 3. The stem of the cap 1102 may have one or more longitudinal grooves that allow the disinfectant in the cap 1102 to pass the stem to contact the exterior surfaces of the needleless valve 1106. FIG. 12 illustrates the medical disinfectant cap 1102 as connected to the 3-way stopcock 1104.

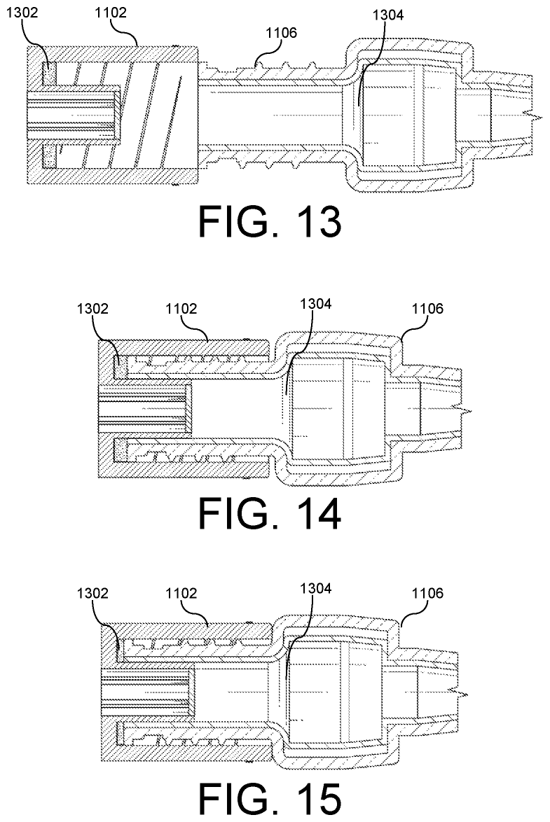

FIGS. 13-15 are cross-sections of the medical disinfectant cap 1102 and the needleless valve 1106 of FIGS. 11 and 12. In each of FIGS. 13-15, an annular sponge 1302 is disposed within the cap 1102. In FIG. 13, the cap 1102 and the needleless valve 1106 have been brought into contact, but their threads have not engaged.

In FIG. 14, the cap 1102 has been threaded partially onto the needleless valve 1106, and the needleless valve 1106 has made contact with the sponge 1302.

In FIG. 15, the cap 1102 has been threaded fully onto the needleless valve 1106, which has compressed the sponge 1302, thereby releasing disinfectant to bathe and disinfect the exterior surfaces of the needleless valve 1106, including the surface of the silicone insert 1304 of the needleless valve.

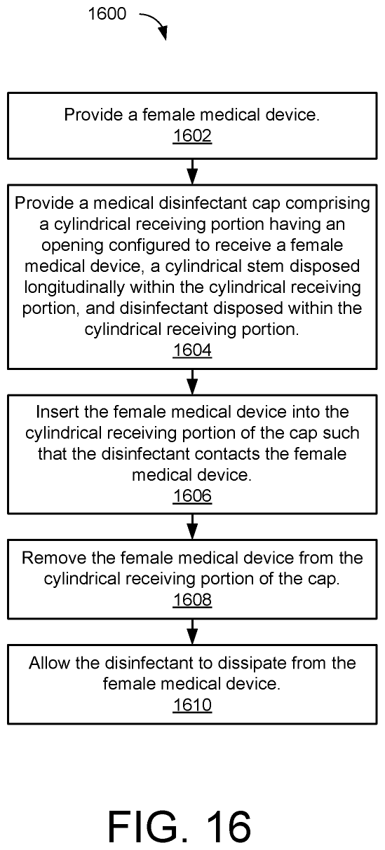

FIG. 16 is a flowchart illustrating a process 1600 for disinfecting a medical device according to some embodiments of the disclosed technologies. For example, the process 1600 may be employed to disinfect the female luer lock 606 of FIG. 6 using the cap 100 of FIG. 1. As another example, the process 1600 may be employed to disinfect the needleless valve 1106 of FIG. 11 using the cap 300 of FIG. 3.

The elements of the process 1600 are presented in one arrangement. However, it should be understood that one or more elements of the process may be performed in a different order, in parallel, omitted entirely, and the like. Furthermore, the process 1600 may include other elements in addition to those presented.

Referring to FIG. 16, the process 1600 may include providing a medical device, at 1602. For example, the medical device may be a female luer lock, a needleless valve, or a similar medical device.

The process 1600 may include providing a medical disinfectant cap, the cap comprising a cylindrical receiving portion having an opening configured to receive a medical device, a cylindrical stem disposed longitudinally within the cylindrical receiving portion, and disinfectant disposed within the cylindrical receiving portion, at 1604.

With continued reference to FIG. 16, the process 1600 may include inserting the medical device into the cylindrical receiving portion of the cap such that the disinfectant contacts the medical device, at 1606. In some embodiments, the cylindrical receiving portion comprises internal threads configured to mate with external threads of the medical device. In these embodiments, inserting the medical device into the cylindrical receiving portion of the cap may include turning the cap with respect to the medical device such that the internal threads of the cap engage external threads of the medical device. In some of these embodiments, the cap includes an annular sponge disposed about the cylindrical stem and contacting the disinfectant. In such embodiments, the process may include turning the cap with respect to the medical device such that the internal threads of the cap engage external threads of the medical device and the annular sponge is compressed to release the disinfectant.

The process 1600 may include removing the medical device from the cylindrical receiving portion of the cap, at 1608, and allowing the disinfectant to dissipate from the medical device, at 1610. For example, the disinfectant may dissipate in approximately one minute. At this point, the medical device is disinfected, and may be connected to the IV system again.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

A person of ordinary skill in the art will recognize that they may make many changes to the details of the above-described memory device without departing from the underlying principles. Only the following claims, however, define the scope of the memory device.

What is claimed is:

1. A cap for disinfecting a medical device, the cap comprising:
a cylindrical receiving portion having an opening configured to receive a medical device, the cylindrical receiving portion comprising internal threads configured to mate with external threads of the medical device;
disinfectant disposed within the cylindrical receiving portion;
a cylindrical stem disposed longitudinally within the cylindrical receiving portion; and
an annular sponge disposed about the cylindrical stem and contacting the disinfectant.

2. The cap of claim 1, wherein the annular sponge is made of polyurethane.

3. The cap of claim 1, wherein the annular sponge is approximately ¼ inch thick.

4. The cap of claim 1, further comprising:
one or more longitudinal grooves formed in the cylindrical stem.

5. The cap of claim 1, further comprising:
a removable seal configured to retain the disinfectant within the cylindrical receiving portion.

6. The cap of claim 1, wherein the medical device comprises one of:
a female luer lock; or
a needleless valve.

7. A cap for disinfecting a medical device, the cap comprising:
a cylindrical receiving portion having an opening configured to receive a medical device;
a cylindrical stem disposed longitudinally within the cylindrical receiving portion; and
disinfectant disposed within the cylindrical receiving portion.

8. The cap of claim 7, wherein the cylindrical receiving portion comprises:
internal threads configured to mate with external threads of the medical device.

9. The cap of claim 7, further comprising:
an annular sponge disposed about the cylindrical stem and contacting the disinfectant.

10. The cap of claim 9, wherein the annular sponge is made of polyurethane.

11. The cap of claim 9, wherein the annular sponge is approximately ¼ inch thick.

12. The cap of claim 7, further comprising:
one or more longitudinal grooves formed in the cylindrical stem.

13. The cap of claim 7, further comprising:
a removable seal configured to retain the disinfectant within the cylindrical receiving portion.

14. The cap of claim 7, wherein the disinfectant is 70/30 isopropyl alcohol.

15. The cap of claim 7, wherein the medical device comprises one of:
a female luer lock; or
a needleless valve.

16. A method of disinfecting a medical device, the method comprising:
providing a medical device;
providing a cap, the cap comprising:
a cylindrical receiving portion having an opening configured to receive a medical device,
a cylindrical stem disposed longitudinally within the cylindrical receiving portion, and
disinfectant disposed within the cylindrical receiving portion; and
inserting the medical device into the cylindrical receiving portion of the cap such that the disinfectant contacts the medical device.

17. The method of claim 16, wherein the cylindrical receiving portion comprises internal threads configured to mate with external threads of the medical device, the method further comprising:
turning the cap with respect to the medical device such that the internal threads of the cap engage external threads of the medical device.

18. The method of claim 17, wherein the cap comprises an annular sponge disposed about the cylindrical stem and contacting the disinfectant, the method further comprising:
turning the cap with respect to the medical device such that the internal threads of the cap engage external threads of the medical device and the annular sponge is compressed to release the disinfectant.

19. The method of claim 16, further comprising:
removing the medical device from the cylindrical receiving portion of the cap; and
allowing the disinfectant to dissipate from the medical device.

20. The method of claim 16, wherein the medical device comprises one of:
a female luer lock; or
a needleless valve.
